(19) United States
(12) Patent Application Publication
(10) Pub. No.: US 2023/0126580 A1
(43) Pub. Date: Apr. 27, 2023

(54) APPARATUS AND METHOD FOR RAPID MONITORING OF PATHOGENS IN WATER

(71) Applicant: CRANFIELD UNIVERSITY, Bedfordshire (GB)

(72) Inventor: Zhugen YANG, Bedfordshire (GB)

(21) Appl. No.: 17/801,677

(22) PCT Filed: Aug. 18, 2021
(86) PCT No.: PCT/GB2021/052136

§ 371 Date: Aug. 23, 2022

(30) Foreign Application Priority Data
Aug. 25, 2020 (GB) 2013248.6

(51) Int. Cl. C12Q 1/689; B01L 3/00; C12Q 1/6806

(52) U.S. Cl. CPC C12Q 1/689; B01L 3/50273; B01L 3/5023; C12Q 1/6806; B01L 2200/10; B01L 2300/0825; B01L 2300/126; B01L 2400/0406

(57) ABSTRACT

A method of detecting a pathogen in a water sample. The method includes extracting DNA of the pathogen from a DNA solution derived from a water sample, eluting the DNA through a paper-based microfluidic device having flow channels and then performing LAMP reactions within a set of reaction chambers to obtain LAMP products that may be detected via an amplicon detection test.

### FIELD OF INVENTION

The present invention relates to a device and method for detecting a pathogen in water and in particular, although not exclusively, to a system for rapid monitoring of a pathogen in a water system such as a wastewater flow.

### BACKGROUND

The novel coronavirus pneumonia (COVID-19) caused by SARS-CoV-2 infection spread rapidly around the globe. Although public health authorities raced to contain the spread of the virus, cumulative deaths escalated. Some clinical studies found that some carriers of the virus are asymptomatic, with no fever, and no, or only slight symptoms of infection. Without the ability to screen asymptomatic patients quickly and effectively, these unsuspecting carriers have the potential to increase the risk of disease transmission if no early effective quarantine measures are implemented.

Accordingly, fast and accurate screening of potential virus carriers and diagnosis of asymptomatic patients would be an important step for intervention and prevention at an early stage. It remains a highly challenging logistical exercise for medical professionals to practically and effectively screen suspected infectious cases from individual households. Such an undertaking is time-consuming, labour intensive and is constrained by the availability of testing technologies.

Paper analytical devices have emerged as powerful tools for the rapid diagnosis of pathogens. These origami-type devices are small analytical tools having different functional paper areas or regions that may be created by printing a hydrophobic wax on to the paper using a wax printer. The devices integrate the various processes of extraction, enrichment, purification, elution, amplification, and visual detection that are required, for example, for nucleic acid testing. The testing process may be undertaken through simple folding of a paper-based device in different ways and the eluting of fluids through the device without a pump or power supply. The paper analytical devices enable multiplexed sensitive assays that rival polymerase chain reactions (PCR) laboratory-based assays to provide high-quality, fast precision diagnostics for pathogens. For example, a study has demonstrated the multiplexed determination of malaria from whole blood using a paper-based device in rural Uganda [Reboud, J.; Xu, G.; Garrett, A.; Adriko, M.; Yang, Z.; Tukahebwa, E. M.; Rowell, C.; Cooper, J. M. ‘Paper-based microfluidics for DNA diagnostics of malaria in low resource underserved rural communities’, Proc. Natl. Acad. Sci. U.S.A 2019, 116 (11), 4834-4842]. The test could sensitively analyse multiplexed nucleic acid sequences of pathogens within 50 min, which gave a higher-quality and faster precision diagnosis for malaria than PCR. In addition, paper analytical devices are easy to stack, store, and transport as they are thin and lightweight. Visual analysis is made simple due to the strong contrast with a coloured substrate. These paper-based devices can also be incinerated after use, reducing the risk of further contamination. However, such earlier systems have utilised direct biological material samples which would require the abovementioned time-consuming and labour-intensive screening to collect an individual's blood or saliva. Accordingly, what is required is apparatus, a method and system that addresses these problems.

### SUMMARY OF THE INVENTION

It is an objective of the present invention to provide a system for rapid analysis of pathogens such as microorganisms in water to enable early detection. It is a further specific objective to provide a device and method for detection of pathogens, microorganisms, infectious diseases, bacteria, a virus and the like within a water network such as a wastewater flow from a commercial or domestic building.

It is a yet further specific objective to provide an earlier identification and detection system for high infectious diseases including in particular coronavirus species in wastewater from buildings including community wastewater, so as to identify affected households, communities, local populations and to minimise pathogen spread and the risk to public health.

Accordingly, the subject invention provides an early warning system that includes a rapid analytical device and method for on-site detection of viruses in wastewater. The present system utilizes wastewater-based epidemiology (WBE), to provide an effective approach to predict the potential spread of the infection by testing for infectious agents in wastewater. The present system also finds application as an effective means to trace illicit drugs, obtain information on health, disease, and pathogens at a local population/community level.

Faeces and urine from disease carriers in a community will contain many biomarkers that can enter the sewer system. The coronavirus infection disease (SARS-CoV-2) is capable of being isolated from faeces and urine of infected people and it has been shown to survive for up to several days in an appropriate environment after exiting the human body. The present device and method is directed to the monitoring, detection and analysis of infectious disease such as coronavirus species e.g., COVID-19, in household and community wastewater so as to trace the pathogen sources through sewage pipe networks and determine whether there are potential pathogen carriers in certain local areas. The present system provides infectious diseases monitoring at a community/household level and at a very early stage through WBE thereby enabling effective intervention such as movement restrictions on local populations to minimize spread of the pathogen.

The inventors provide an efficient, transportable and robust analytical tools to accurately and quickly identify trace or low-level pathogen sources through WBE so as to screen asymptomatic infected cases without centralized laboratories. The present WBE early warning and intervention system utilises a rapid analytical method and device for on-site detection of viruses in water. In particular, the inventors provide a microfluidic cellulose/paper-based analytical device (μPAD) to detect pathogens in a water sample such as a sample of wastewater from a household or community wastewater outlet. The presently developed monitoring and detection tool provides a fast ‘sample-to-answer’ analysis system for quantitative monitoring of nucleic acids and genetic information through the analysis of sewage. The present μPADs are small and portable and can detect pathogens in wastewater on site.

The present μPADs provide a substrate with a plurality of microfluidic channels and reaction chambers that is inexpensive, lightweight, disposable, and can be manufactured conveniently and readily. Cellulose/paper is a suitable construction material for the present μPADs due to its physical characteristics and in particular its hydrophilicity and capability to allow various solutions to flow through its porous structure via capillary action.

The present μPADs and methods are biocompatible with biological samples and may be used with a variety of different sensing mechanisms such as colorimetric, electrochemical, chemiluminescence (CL), electro-chemiluminescence (ECL) and other signal detection with the results being used quantitatively and/or quantitatively for diagnostic testing. Such sensing mechanisms, techniques and methods may be integrated with the present μPADs specifically for the detection of pathogens. Additionally, such systems enable bioassays to be undertaken and the results obtained simultaneously. Moreover, the present μPADs and methods are compatible for use with a digital camera or camera-enabled phone (smart phone) to collect data and images conveniently. Such data may then be used directly (or locally) or transmitted wirelessly over communications networks to centralized laboratories for analysis and results processing in real-time.

According to a first aspect of the present invention there is provided a method of detecting a pathogen present in a water sample comprising: extracting nucleic acid of the pathogen from a nucleic acid solution derived from the water sample at a solid phase extraction structure mounted at a first layer of a multilayer device; eluting nucleic acid from the solid phase extraction structure to at least a second layer of the multilayer device having paper-based fluid flow channels; allowing the nucleic acid to flow thought the paper-based fluid flow channels to a further layer of the multilayer device having discrete reaction chambers, each of the chambers fed respectively by at least one of the fluid flow channels; performing LAMP reactions within each reaction chamber to obtain LAMP products; and detecting the LAMP products via an amplicon detection test.

Reference within the specification to the extraction, elution, flow etc of nucleic acid or a nucleic acid-based analogue includes the extraction, elution, flow etc of compounds having nucleotide repeating units such as DNA, RNA and/or nucleic acid analogues of a pathogen.

Optionally, the method comprises filtering the water sample through a filter membrane and adding a lysis buffer to the filtered sample to form the nucleic acid solution. Optionally, prior to said step of eluting the nucleic acid, the method comprises washing the nucleic acid at the solid phase extraction structure with a washing buffer. Optionally, the solid phase extraction structure comprises glass fibre or magnetic beads onto cellulous paper.

Optionally, the step of allowing the nucleic acid to flow comprises allowing the nucleic acid to flow along the paper-based fluid flow channels of the second layer into paper-based fluid flow channels of a third layer positioned adjacent the second layer.

Optionally, the method comprises allowing the nucleic acid to flow from the paper-based fluid flow channels of the third layer into paper-based fluid flow channels of a fourth layer positioned adjacent the third layer.

Optionally, the flow of the nucleic acid in the paper-based fluid flow channels is divided as it transfers between the respective layers.

Optionally, the discrete reaction chambers at the further layer comprises paper inserts positioned within respective holes in the third layer, the further layer comprising a plastic material.

Optionally, prior to said step of performing the LAMP reactions, the method comprises sealing the nucleic acid within the discrete reaction chambers by coating a film onto the further layer to cover the paper inserts within the holes. Optionally, the step of performing the LAMP reactions comprises adding at least one set of LAMP primers to the discrete reaction chambers to create respective LAMP assays. Optionally, the method comprises adding a plurality of different sets of LAMP primers to the discrete reaction chambers. Optionally, the step of performing the LAMP reactions further comprises heating the further layer and the LAMP assays at a predetermined temperature and for a predetermined time. Optionally, the predetermined temperature is in a range 40 to 80° C.; 50 to 75° C.; 55 to 75° C.; or 60 to 70° C. and the predetermined time is in a range 10 to 90 minutes; 20 to 60 minutes; 30 to 50 minutes; or 35 to 55 minutes.

Optionally, the step of detecting the LAMP products comprises monitoring and capturing a UV or colorimetric signal from the LAMP products emitted from the reaction chambers. Optionally the signal may be fluorescence, colorimetric or UV based. Optionally, the captured images are UV-torch luminated signals. Optionally, the signal emitted by the sample is a fluorescent signal.

Optionally, the step of capturing the UV signal comprises recording the fluorescent UV signal as a photographic image. Optionally, the method comprises analysing the at least one photographic image using software to obtain an average UV intensity of the LAMP products emitted from the respective reaction chambers. Optionally, the step of detecting the LAMP products comprises using one of the discrete reaction chambers as an internal positive control containing a predetermined genomic nucleic acid as a template and using one of the discrete reaction chambers as an internal negative control containing a predetermined genomic nucleic acid as a template. Optionally, the method comprises normalising the average signal intensity of the LAMP products using an average intensity of the positive control and the negative control respectively.

According to a second aspect of the present invention there is provided a multilayer device for detecting a pathogen present in a water sample comprising: a sample preparation part having at least one layer including a solid phase extraction structure mounted therein to receive a nucleic acid solution derived from the water sample; a fluid flow part comprising a plurality of layers each having paper-based fluid flow channels therein to enable fluid capillary flow from the solid phase extraction structure through the plurality of layers; and a reaction layer comprising a plurality of discrete reaction chambers each provided in fluid communication with the fluid flow channels to receive by capillary flow a fluid from the fluid flow channels.

Optionally, the device comprises a filter membrane positioned in a fluid flow direction upstream of the solid phase extraction structure to enable a pre-filtering of the water sample and a nucleic acid lysising of the pathogen to form the nucleic acid solution.

Optionally, the plurality of layers of the fluid flow part comprises a plurality of primary layers each of the primary layers divided into a plurality of secondary layers. Optionally, the primary layers and the secondary layers are integrally formed and coupled to one another by folded or hinge regions positioned at respective edges of the primary and secondary layers.

Optionally, the fluid flow channels within each primary layer are divided respectively at the folded or hinge regions that divide respectively the primary layers into the secondary layers.

Optionally, the sample preparation part further comprises a layer having a sample introduction port and a layer having a waste collection component. Optionally, the reaction layer comprises a plastic material having a plurality of holes and paper inserts positioned within the holes to define the discrete reaction chambers.

Optionally, the device comprises at least one set of LAMP primers for introduction to the discrete reaction chambers.

Optionally, the device comprises a lateral flow device having a plurality of lateral flow detection strips in fluid communication with the discrete reaction chambers respectively.

Optionally, the device comprises a camera to capture an image of the discrete reaction chambers and software to analyse the image captured by the camera. Optionally, the software is configured to analyse the images captured by the camera to determine an average intensity (fluorescence or UV) generated by the LAMP products derived from the LAMP primers.

The present pPADs may be fabricated by various methods, such as photolithography, inkjet printing, polydimethylsiloxane (PDMS) plotting, wax printing, wax dipping, wax screen printing and plasma treatment.

According to a further aspect of the present invention there is provided a use of the method and/or device as claimed herein to detect a pathogen in a water sample. Optionally, the pathogen is a microbe, an infectious disease, a bacteria or a virus. Optionally, the infectious disease is a coronavirus and optionally COVID-19.

### BRIEF DESCRIPTION OF DRAWINGS

A specific implementation of the present invention will now be described, by way of example only, and with reference to the accompanying drawings in which:

FIG. 1 is a schematic of a microfluidic paper-based analytical device according to one aspect of the present invention having a plurality of paper-based layers or plates connected together via folded regions;

FIG. 2 is a schematic illustration of a method of manufacture of the microfluidic device of FIG. 1;

FIG. 3 is a schematic flow diagram of the various stages of a paper-based loop mediated isothermal amplification (LAMP) assay system for detection of a pathogen using the device of FIG. 1;

FIG. 4 are photographs of the LAMP reaction plate and chambers of the device of FIG. 1 after the LAMP reactions using the device of FIG. 1 and analytical steps of FIG. 3;

FIG. 5 is a schematic illustration of various sample processing steps according to one aspect of the present method and apparatus;

FIG. 6A is an agarose gel image of PCR reaction results for the detection of three genomic DNA targets extracted from cultured organisms;

FIG. 6B is an agarose gel image of LAMP reaction results for the detection of three genomic DNA targets under negative control;

FIG. 7A illustrates a specific implementation of a paper-based analytical device according to one aspect of the present invention;

FIG. 7B is a graph of normalised intensity versus cycle threshold of a paper-based PCR assay of Brucella genomic DNA at different pore sizes;

FIG. 7C is a graph of cycle threshold versus diameter or pore/flow channel of the present paper device;

FIG. 7D is a graph of normalised intensity versus cycle threshold of a paper-based PCR assay of Brucella genomic DNA for different flow channel widths;

FIG. 7E is a graph of cycle threshold versus channel width of the present paper device;

FIG. 7F is a graph of normalise intensity versus cycle threshold of a paper-based PCR assay of Brucella genomic DNA for different flow channel lengths;

FIG. 7G is a graph of cycle threshold versus flow channel length the present paper device;

FIG. 8A is an image of LAMP assay products at various concentration ratios of Calcein to Mn²⁺ for LAMP assay;

FIG. 8B is a graph of the relationship between time and concentration ratio of Calcein to Mn²⁺;

FIG. 8C is a graph of the relationship between the respective fluorescence intensity ratios of a positive control and a negative control and the concentration ratio of Calcein to Mn²⁺;

FIG. 9A is a graph of normalised intensity versus cycle time of a paper-based PCR assay of Brucella genomic DNA with the present paper-based device;

FIG. 9B is a graph of normalised intensity and cycle number of the results of a paper-based PCR assay of Brucella genomic DNA with the paper-based device;

FIG. 9C is a graph of cycle time versus number of layers of a paper-based PCR assay of Brucella genomic DNA;

FIG. 10A is a graph of normalised intensity versus time of Real-time LAMP-assay for the detection of model organism Brucella;

FIG. 10B is a graph of time versus Brucella of Real-time LAMP assay for the detection of model organism Brucella;

FIG. 10C is a graph of normalised intensity versus time of the results of a paper-based LAMP assay for Brucella detection;

FIG. 10D is a graph of time versus Brucella of the results of a paper-based assay for Brucella detection;

FIG. 11A is a graph of time versus Salmonella FIP/BIP concentration of a Real-time LAMP assay;

FIG. 11B is a graph of time versus E. coli FIP/BIP concentration of a Real-time LAMP assay;

FIG. 11C is a graph of time versus C. perfringens FIP/BIP concentration of a Real-time LAMP assay;

FIG. 12A is a graph of normalised intensity versus time of the results of real-time LAMP assay on Salmonella genomic DNA;

FIG. 12B is a graph of normalised intensity versus time of the results of real-time LAMP assay on Salmonella genomic DNA with various concentrations;

FIG. 12C is a graph of normalised intensity versus time for the results of real-time LAMP assay on E. coli genomic DNA;

FIG. 12D is a graph of normalised intensity versus time of the results of real-time LAMP assay on E. coli genomic DNA with various concentrations;

FIG. 12E is a graph of normalised intensity versus time of the results of real-time LAMP assay on C. perfringens genomic DNA;

FIG. 12F is a graph of normalised intensity versus time of the results of the real-time LAMP assay on C. perfringens genomic DNA with various concentrations;

FIG. 13A is a fluorescence image of the detection of real-time LAMP amplification products of Salmonella genomic DNA at different concentrations;

FIG. 13B is an agarose gel image of a real-time LAMP assay on Salmonella at different concentrations;

FIG. 13C is a fluorescence image of the detection of real-time LAMP amplification products of E. coli at different concentrations;

FIG. 13D is an agarose gel image of a real-time LAMP assay on E. coli at different concentrations;

FIG. 13E is a fluorescence image of the detection of real-time LAMP amplification products of C. perfringens at different concentrations;

FIG. 13F is an agarose gel image of a real-time LAMP assay of C. perfringens;

FIG. 14A is a fluorescence image of a Real-time LAMP assay for Salmonella genomic DNA primers set;

FIG. 14B is an amplification curve plot of real-time LAMP assay with Salmonella primers set;

FIG. 14C is a fluorescence image of a Real-time LAMP assay for E. coli primers set;

FIG. 14D is an amplification curve plot of real-time LAMP assay with E. coli primers set;

FIG. 14E is a fluorescence image of a Real-time LAMP assay with C. perfringens primers set;

FIG. 14F is an amplification curve plot of real-time LAMP assay with C. perfringens primers set;

FIG. 15A is a graph of normalised intensity versus time for a paper-based LAMP assay on Salmonella genomic DNA at various concentrations;

FIG. 15B is a graph of the results of a paper-based LAMP assay on Salmonella genomic DNA at various concentrations;

FIG. 15C is a graph of normalised intensity versus time for a paper-based LAMP assay on E. coli genomic DNA at various concentrations;

FIG. 15D is a graph of the results of a paper-based LAMP assay on E. coli genomic DNA at various concentrations;

FIG. 15E is a graph of normalised intensity versus time for a paper-based LAMP assay on C. perfringens genomic DNA at various concentrations;

FIG. 15F is a graph of the results of a paper-based LAMP assay on C. perfringens genomic DNA at various concentrations;

FIG. 16A is a fluorescence image of paper-based LAMP on Salmonella genomic DNA at various concentrations;

FIG. 16B is an agarose gel image of paper-based LAMP assay on Salmonella genomic DNA at various concentrations;

FIG. 16C is a fluorescence image of paper-based LAMP on E. coli genomic DNA at various concentrations;

FIG. 16D is an agarose gel image of paper-based LAMP assay on E. coli genomic DNA at various concentrations;

FIG. 16E is a fluorescence image of paper-based LAMP on C. perfringens genomic DNA at various concentrations;

FIG. 16F is an agarose gel image of paper-based LAMP assay on C. perfringens genomic DNA at various concentrations;

FIG. 17A is fluorescence image of paper-based LAMP assay with Salmonella primers sets;

FIG. 17B is an amplification curve plot of paper-based LAMP assay with Salmonella primers sets;

FIG. 17C is fluorescence image of paper-based LAMP assay with E. coli primers sets;

FIG. 17D is an amplification curve plot of paper-based LAMP assay with E. coli primers sets;

FIG. 17E is fluorescence image of paper-based LAMP assay with C. perfringens primers sets;

FIG. 17F is an amplification curve plot of paper-based LAMP assay with C. perfringens primers sets.

### DETAILED DESCRIPTION OF PREFERRED EMBODIMENT OF THE INVENTION

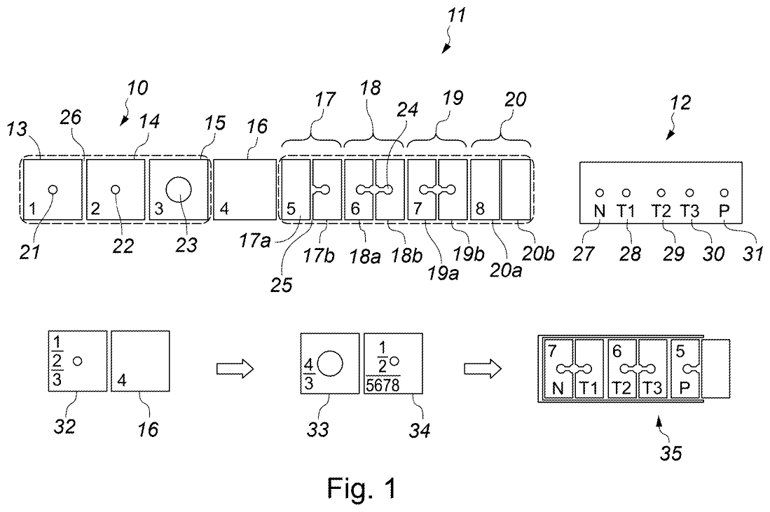

Referring to FIG. 1, a microfluidic cellulose/paper-based analytical device (μPAD) is formed as a multilayer system having generally a three-part configuration including a sample preparation part 10, a fluid flow part 11 and a reaction layer 12. In particular, the device contains three components: a filter paper-based microfluidic device with wax-printed microfluidic channels, a single sided optical adhesive film sealed plastic plate with five loop-mediated outer thermal amplification (LAMP) reaction chambers (N, T1, T2, T3 and P representing 5 reaction chambers, N referring to the internal negative control, T referring to the target, P referring to the internal positive control) and one glass fiber circular disk (4 mm in diameter) for absorbing nucleic acids from the sample. The unfolded paper device has a sample preparation zone and a detection zone. The footprint of the present device is 3 cm×3 cm for each panel (3×24 cm unfolded).

In particular, sample preparation part 10 comprises a multilayer construction having a first layer 13, second layer 14 and third layer 15. The fluid flow part 11 comprises four primary layers 17, 18, 19 and 20 each divided into two secondary layers 17a, 17b; 18a, 18b; 19a, 19b; 20a, 20b, respectively. An intermediate layer 16 provides a bridging layer between the sample preparation part 10 and the fluid flow part 11. Each of the layers 13 to 20 are formed as a folded origami paper-based device with the layers 13 to 20 forming a single paper unit having multiple folds 26 that separate the paper unit into the individual layers 13 to 20. Primary layers 17 to 20 are divided by respective primary folds 24 and are each, in turn, subdivided into the secondary layers 17a to 20b by secondary folds 25. Each of the paper layers 13 to 20 (alternatively termed panels, plates or sheets), that form the present μPAD are constructed from a paper strip having dark regions that are created by printing the paper with hydrophobic wax leaving non-waxed paper regions that represent fluid flow channels. In particular, the fluid flow part 11 comprises fluid flow channels 24 present within at least some, most or all of the layers 17 to 20 that a conduit to direct sample flow along the layers and from layer to layer from the initial sample preparation part 10 to a final reaction layer 12.

First layer 13 comprises a glass fibre disc 21 mounted within the wax printed paper construction. The second layer 14 comprises a sample introduction port 22. Third layer 15 comprises a waste zone 23 defined as a region of blotting paper surrounded by the dark region (hydrophobic wax).

Reaction layer 12 is formed as a plastic plate and comprises a plurality of holes 27 to 31 each accommodating a paper insert (or spot). These holes/paper inserts define respective reaction chambers provided in fluid communication with the fluid flow channels 24 of part 11. Accordingly, a fluid sample introduced into port 22 is configured to flow onto the solid phase extraction structure (defined by glass fibre disc 21) and into the fluid flow channels 24. Within the fluid flow part 11, the fluid flows under capillary action and the flow path is divided or split at each region of the secondary folds 25. The flow is eventually feds into the respective reaction chambers 27 to 31.

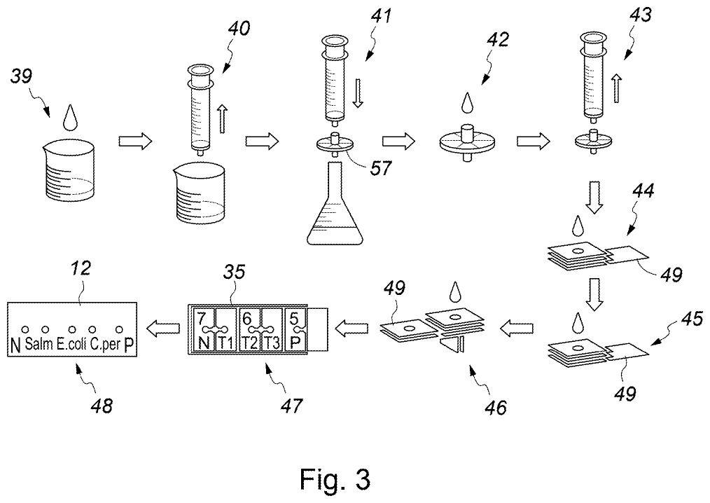

As illustrated in FIG. 1, the paper strip that forms the multilayer μPAD is folded so as to stack the layers 17 to 20 on top of one another with the plastic plate 12 coupled to the sample preparation part 10 and fluid flow part 11 so as to represent a terminal end of the μPAD. Layer stack 32 illustrates the layers folded in a certain configuration to receive a water sample containing a pathogen for detection and analysis. The μPAD may then be refolded with the layers presented in a different order referring to stack 33 and 34 as a water sample is processed through the microfluidic device to the reaction chambers 27 to 29 as described further with reference to FIG. 3.

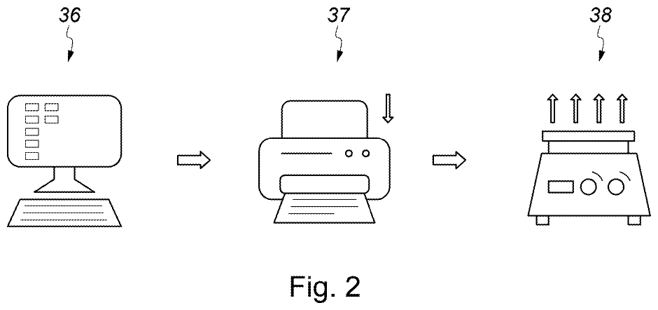

Referring to FIG. 2, the present μPAD was fabricated using a computer-based drawing package (ColeDraw) at stage 36. The paper strip, divided into the multiple layers 13 to 20, was printed by wax-printing to define the various mounting (or receiving) holes and the fluid flow channels at stage 37. The resulting multi-panel construction was then baked on a hot plate at 130° C. for 5 min to melt the printed wax at stage 38. The wax could penetrate through the paper to form the hydrophobic area due to the porous structure of the cellulose filter paper. On the contrary, the unpatterned area retained good hydrophilicity. The wax-penetrated paper was cut into individual devices for further LAMP experiments.

To assess the analytical sensitivity and suitability of the present paper-based device, three target bacteria were investigated: Salmonella; E. coli and C. perfringens. FIG. 3 illustrates schematically a flow diagram of the various steps of the present real-time loop-mediated outer thermal amplification (LAMP) technique for pathogen detection in a water sample using the present μPAD.

Referring to FIG. 3, certain volumes of each bacteria were spiked into tap water at stage 39 followed by the absorption of the spiked tap water at stage 40. At stage 41, the bacteria containing water sample was processed through a filter membrane 57 (i.e., 25 μL of the sample solution was introduced to the glass fiber which was forced through a 4 mm hole punched into the printed panel). The sample solution penetrated through the glass fiber and was later absorbed by the large hydrophilic disk on the third panel of the paper-based device by capillary action. The DNA was captured by the glass fiber during this process. The cell residue was rinsed off using 25 μL of washing buffer (70% ethanol). Afterwards, the paper device was folded for elution.

Specifically, a lysis buffer was added of at stage 42. At stage 43, the DNA solution was then absorbed and introduced onto the present μPAD 47 at stage 44. This was followed by DNA washing using 25 μL of a washing buffer (70% ethanol) at stage 45 and then DNA elusion (using a DNA elusion buffer) at stage 46. The DNA solution containing the extracted DNA was then allowed to progress through part 11 of the μPAD 47 under the capillary flow (via fluid flow channels 24) where the flow was divided at stage 47 according to the folded configuration 35. The extracted DNA was then delivered to the reaction part 12 at stage 48 and into the respective reaction chambers 27 to 31. A set of loop-mediated outer-thermal amplification (LAMP) primers were then pipetted into the respective reaction chambers 27 to 31 to create the respective LAMP assays and to generate the LAMP products (amplicons). The reaction chambers 27 to 39 where heated at 65° C. on a hot plate to perform the multiplex LAMP reactions.

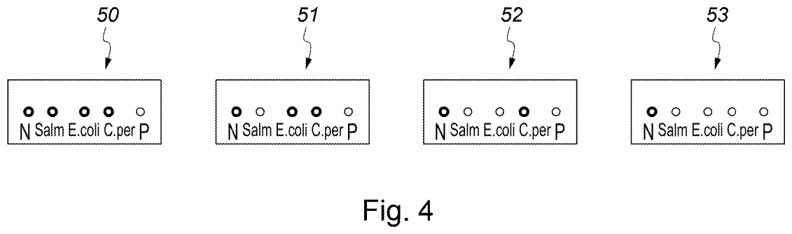

In one embodiment, fluorescence was used as the detection method. Referring to FIG. 4, four sets of results 50,51, 52, 53 are presented detailing the fluorescence signals from the reaction chamber 27 to 31 of the plastic plate 12. The labels indicate different species-specific LAMP reaction. N: an internal negative control; P: an internal positive control; Salm (Salmonella); E. coli (E. coli); C. per (C. perfringens). The results are the representative images for single (Salmonella), duplex (Salmonella, E. coli), and triplex (Salmonella, E. coli, C. perfringens) target detection, together with the negative control and the positive control to determine the effectiveness of the test (green colour was observed for P and no green colour was observed for N). The fluorescence images was captured and recoded using a digital camera, in particular a mobile phone camera.

Experimental

Materials

Whatman chromatography paper No. 1 (pure cellulose paper) purchased from GE Healthcare Worldwide (UK) was wax printed by a Xerox ColorQube 8580 digital wax printer from Xerox (UK). The Black Cast Acrylic obtained from Stockline Plastics (UK) was processed by a Laser cutter from Laserscript (UK). A Bio-Rad C1000 Thermal Cycler, a horizontal electrophoresis apparatus and a Gel Doc XR+ Imager for PCR assay and LAMP assay were from Bio-Rad Laboratories (UK). The hot plate, the Digital Drybath and the UV LAMP (366 nm) were purchased from Fisher Scientific (UK). The MicroAmp Optical Adhesive Film was from Thermo Scientific (UK), and the punchers were from kai Europe GmbH (Germany). PCR Master Mix and LAMP Master Mix were purchased from Agilent Technologies (UK) and OptiGene (UK), respectively. Evergreen was from Cambridge BioScience (UK), while Calcein, Manganese (II) chloride solution and double distilled H2O (ddH2O) were from SIGMA (UK). A MagaZorb DNA Mini-Prep Kit was from Promega (UK) and a Nucleopore DNA isolation Mini Kit was from Genetix (India). A Qubit 2.0 Fluorometer was from Thermo Fisher Scientific (UK). LAMP primers used in this work were synthesized by Eurofins (Germany). Bacterial strains of Salmonella, E. coli and C. perfringens were isolated and kindly supplied by the Scottish Water staff.

Loop Mediated Isothermal Amplification (LAMP) Assay

The optimization of the LAMP assay was performed on a Bio-Rad C1000 Thermal Cycler. The LAMP amplicons were analyzed on 3% agarose gel in 1×TAE buffer and the related image was recorded by a Gel Doc XR+ Imager (Figure S2). The LAMP primers sets for Salmonella, E. coli, C. perfringens and Brucella are detailed in Table S1. Besides LAMP primers, the 20 μL reaction mixture of LAMP assay also contains 0.4 mM dNTPs, 4.0 mM MgSO4, 1 M betaine, Ibuffer (20×), 25 μM calcein, 500 μM MnCl₂, 0.4 U Bst Polymerase, 1 μL ddH2O and 2 μL DNA sample. Brucella genomic DNA was chosen as the target of the internal positive control while ddH2O was used as the template of the internal negative control with the same composition.

TABLE 1
LAMP primers

| Target | primers | Sequence (5’-3’) |
|---|---|---|
| Salmonella | SM F3 | GGCGATATTGGTGTTTATGGGG |
| | SM B3 | AACGATAAACTGGACCACGG |
| | SM FIP | GACGACTGGTACTGATCGATAGTTTTTCAACGT |
| | | TTCCTGCGG |
| | SM BIP | CCGGTGAAATTATCGCCACACAAAACCCACCG |
| | | CCAGG |
| | SM LPF | GACGAAAGAGCGTGGTAATTAAC |
| | SM LPB | GGGCAATTCGTTATTGGCGATAG |
| Escherichia coli | EC F3 | GCCATCTCCTGATGACGC |
| | EC B3 | ATTTACCGCAGCCAGACG |
| | EC FIP | CATTTTGCAGCTGTACGCTCGCAGCCCATCATG |
| | | AATGTTGCT |
| | EC BIP | CTGGGGCGAGGTCGTGGTATTCCGACAAACAC |
| | | CACGAATT |
| | EC LPF | CTTTGTAACAACCTGTCATCGACA |
| | EC LPB | ATCAATCTCGATATCCATGAAGGTG |
| Clostridium | CLO F3 | AGATTGATGGAACAGGAACT |
| perfringens | CLO B3 | TTATTATCTGTATCAGGATCCCA |
| | CLO FIP | CACTTTCTGGTTCATTTTTGGACAGCATGCTAT |
| | | GATTGTAACTCAAGG |
| | CLO BIP | AAGAGAACATGCATGAGCTTCACATATGCATTC |
| | | TTATCATAATCTGG |
| | CLO LPF | ATGCTATGATTGTAACTCAAGG |
| | CLO LPB | CATATGCATTCTTATCATAATCTGG |
| Brucella | BCSP F3 | TGGATGAGCCGGGTTCTG |
| | BCSP B3 | GGAACGAGCGAAATACCGT |
| | BCSP FIP | GTCCCGGCTTCAGGTGTTCAGATCGTTCTTGA |
| | | AGCCTACGG |
| | BCSP BIP | GGCGAGAGGCTGAAAGATGGTGCCAGTTCCG |
| | | AGATTGCG |
| | BCSP LPF | CCTTGATATCGTCTTCCGTGAGG |
| | BCSP LPB | TGGACGCCTATTTCTTTGTGG |

To confirm amplification of target sequence in Salmonella, Escherichia coli and Clostridium perfringens, the outer primers (F3 and B3) of each designed LAMP primer set were used for conventional PCR assay. DNA extracted from three organisms were subjected to amplification at a final volume of 20 μl containing 10 μl qPCR Master Mix (Agilent Technologies, UK), 0.2 μM of each primer and 2 μL of template DNA. Amplification cycles consisted of an initial denaturation step at 95° C. for 3 min, followed by 40 cycles of denaturation at 95° C. for 10 s, annealing at 55° C. for 30 s, an extension at 72° C. for 30 s. The PCR amplicons were analysed on 3% agarose gel electrophoresis and visualized in Gel Doc XR+ System (Bio-Rad, USA).

Results

The results of LAMP reactions were read out with a hand-held UV LAMP after incubation in a digital drybath at 65° C. In a LAMP assay, Calcein is used as a colorimetric indicator. Calcein molecules combine with Mn²⁺ before LAMP reaction, quenching calcein fluorescence. As LAMP reaction proceeds in the presence of target DNA, Mn²⁺ complexes with newly generated P₂O₇⁴⁻, therefore calcein molecules recover green fluorescence. Moreover, calcein molecules will combine with residual Mn²⁺, enhancing green fluorescence signal. Eventually, the positive result can be determined from the color change of the LAMP reaction solution from yellow to green by the naked eye. Results can also be read out by a hand-held UV LAMP or digitally collected by a mobile-phone camera.

From previous investigations by the inventors, in Real-time PCR assay, a positive reaction is detected by the accumulation of a fluorescent signal. The Cₜ (cycle threshold) is defined as the number of cycles required for the fluorescent signal to cross the threshold. Therefore, the smaller Cₜ is, the more efficient the reaction is. In addition, Cₜ levels are inversely proportional to the amount of target nucleic acid, and a smaller Cₜ results in a higher DNA yield. It was noted, Cₜ decreases when the pore size increases (from 3.0 mm to 4.0 mm), given that a larger pore size results in a stronger adhesion ability and thus more DNA is attached to the sample pore and a higher DNA yield leads to a smaller Cₜ. However, when the pore size becomes too large, the DNA on the sample pore may not elute entirely and remain on the paper, which in turn causes Cₜ to become large (from 4.0 mm to 5.0 mm). With regard to channel width, a wider channel width caused a lower yield and a larger Cₜ. When fixing the pore size and the channel width, more DNA is attached to the channel due to a longer channel. As the channel length increases (from 1.0 mm to 2.0 mm), the yield decreases and Cₜ becomes larger. Because the difference in Cₜs obtained is quite small, a channel length with a smaller Error Bar and a smaller Cₜ was selected. On the basis of the results it was concluded that the optimal pore size, channel width and channel length of the paper-based device were 4.0 mm, 1.5 mm and 2.5 mm, respectively.

Within the present system and method, LAMP assay Calcein was used as a colorimetric indicator in the place of Evagreen. The concentration ratio of Calcein to Mn²⁺ was optimized by LAMP assay on Brucella DNA. Threshold Time is defined as the time corresponding to 10% of the maximum fluorescence intensity, which is a function of target concentration. The Threshold Time of Real-Time LAMP assay is analogous to the cycle threshold (CO of Real-time PCR assay. Time decreases when the concentration ratio increases, indicating a higher DNA yield. Nevertheless, Ratio (defined as the fluorescence intensity ratio between negative control and positive control) increases with an increasing concentration ratio. When the Ratio is 1:15 and 1:10, the two negative controls show light fluorescence, which will affect the results observed by the naked eye. From the results obtained, the inventors concluded that the optimal concentration ratio of Calcein to Mn²⁺ is 1:20. These optimal parameters were applied to the fabrication of the present μPAD and LAMP experiments.

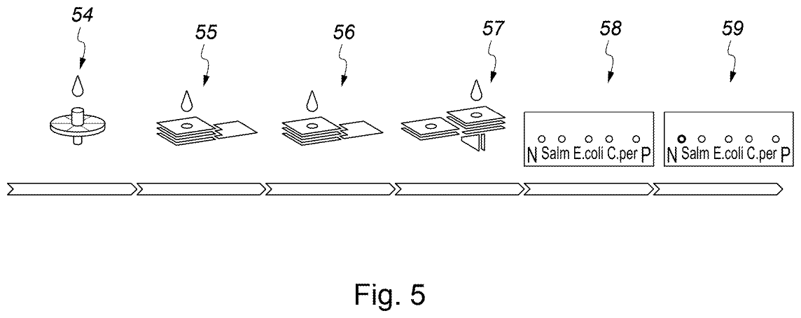

FIG. 5 is a schematic illustration of complete sample processing from initial sample introduction to final bacterial detection. In particular, DNA lysing occurs at stage 54 (5 minutes) followed by sample introduction at stage 55, with extraction occurring after 1 minute. A washing buffer is added at stage 56 for a washing time of 2 minutes followed by addition of an elution buffer at stage 57 with the elusion time period being 2 minutes. The LAMP reaction occurs at stage 58 with the reaction time of 45 minutes. Bacterial detection occurs at stage 59 as indicated in FIG. 5.

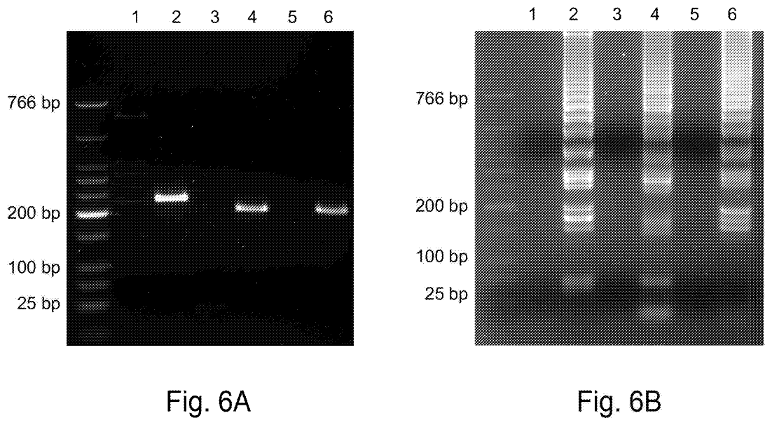

FIGS. 6A and 6B are Agarose gel results of amplification. In particular, FIG. 6A is an Agarose gel image of PCR reaction results for the detection of 3 genomic DNA targets, which were extracted from cultured organisms. 1: ddH2O with Salmonella outer primers, 2: Salmonella amplified with its outer primers, 3: ddH2O with E. coli outer primers, 4: E. coli amplified with its outer primers, 5: ddH2O with C. perfringens outer primers, 6: C. perfringens amplified with its outer primers. FIG. 6B is an Agarose gel image of LAMP reaction results for the detection of 3 genomic DNA targets and a negative control (ddH2O). 1: ddH2O with Salmonella primers sets, 2: Salmonella amplified with its primers sets, 3: ddH2O with E. coli primers sets, 4: E. coli amplified with its primers sets, 5: ddH2O with C. perfringens primers sets, 6: C. perfringens amplified with its primers sets.

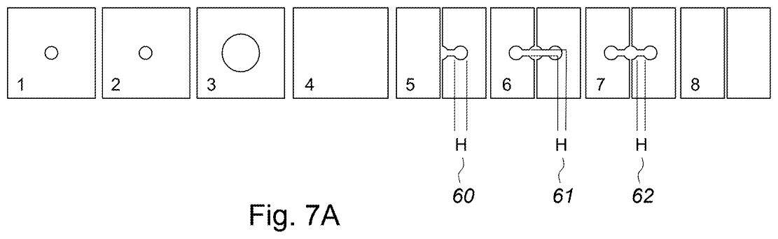

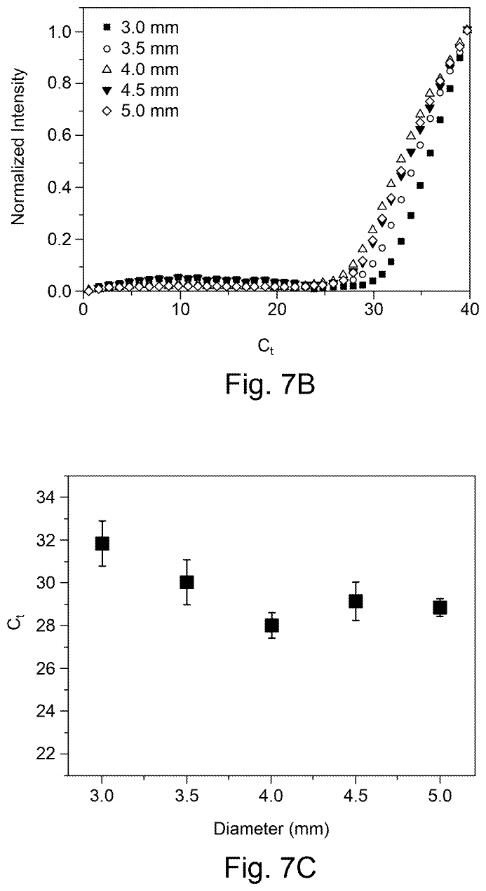

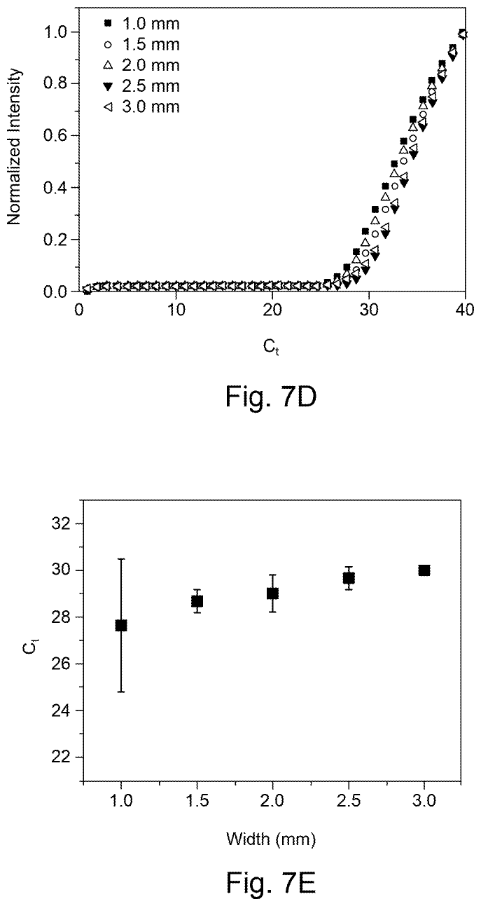

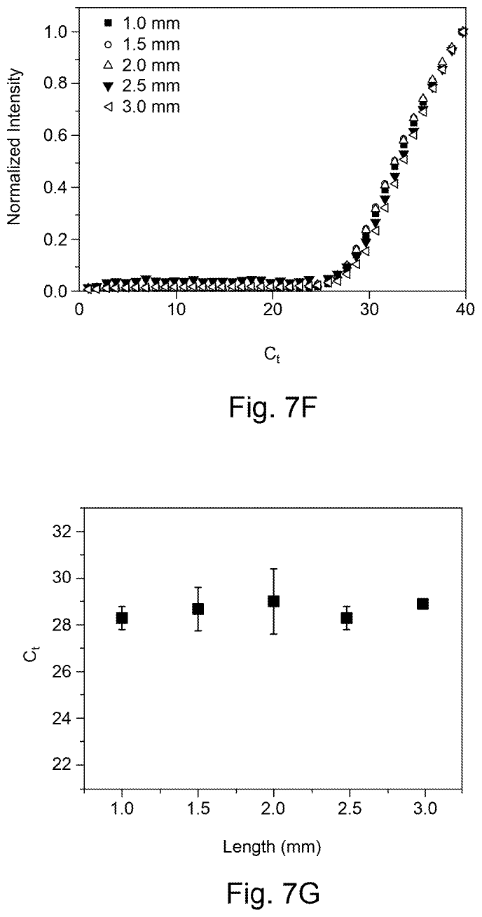

FIG. 7A illustrates optimization of the microfluidic paper-based analytical device. In particular, FIG. 7A is an illustration of the paper device, showing the pore size (diameter 60), channel width 61 and channel length 62.

FIGS. 7B and 7C are graphs of a paper-based PCR assay of Brucella genomic DNA on paper-based device with various pore size (3.0 mm; 3.5 mm; 4.0 mm; 4.5 mm; 5.0 mm), and ddH2O as the negative control.

FIGS. 7D and 7E are graphs of a paper-based PCR assay of Brucella genomic DNA on paper-based device with various channel width (1.0 mm; 1.5 mm; 2.0 mm; 2.5 mm; 3.0 mm). (f) and (g) Paper-based PCR assay of Brucella genomic DNA on paper-based device with various channel length (1.0 mm; 1.5 mm; 2.0 mm; 2.5 mm; 3.0 mm). Cₜ (defined as the number of cycles corresponding to 10% of the maximum fluorescence intensity, Cₜ) as a function of target concentration for Brucella. The optimized results of the pore size, channel width and channel length of the paper-based device are 4 mm, 1.5 mm and 2.5 mm, respectively. The results are averages of three independent experiments and the error bars are the standard deviation.

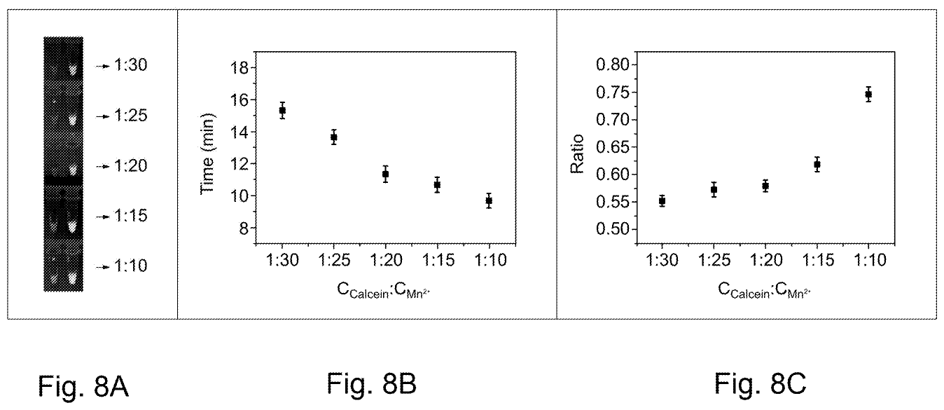

FIGS. 8A to 8C illustrate the optimization of the concentration ratio of Calcein to Mn²⁺ for LAMP assay. In particular, FIG. 8A shows the LAMP assay products with various concentration ratio of Calcein to Mn²⁺ (1:30; 1:25; 1:20; 1:15; 1:10) in ultraviolet light.

FIG. 8B illustrates the relationship between Time and the concentration ratio of Calcein to Mn²⁺. FIG. 8C illustrates the relationship between Ratio (defined as the fluorescence intensity ratio of the positive control and the negative control) and the concentration ratio of Calcein to Mn²⁺.

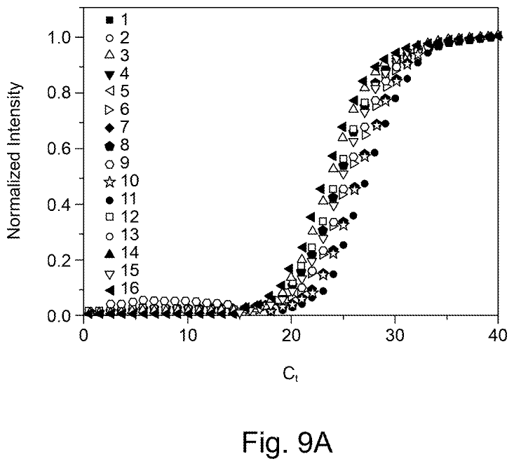

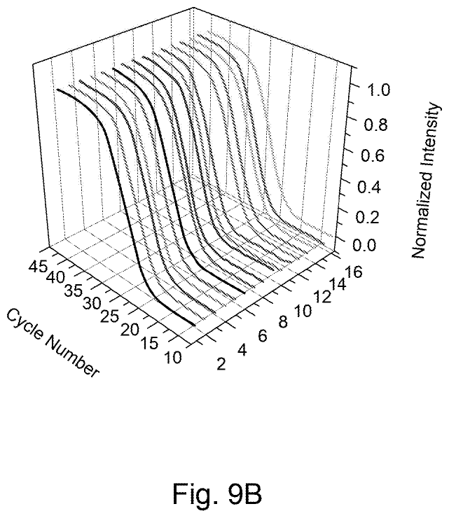

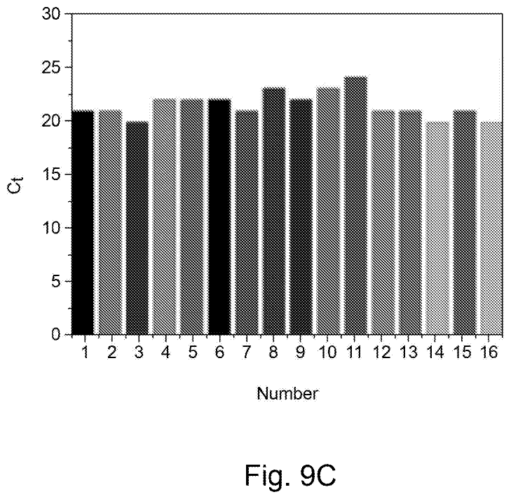

FIGS. 9A to 9C are results of a repeatability test of each layer of the microfluidic paper-based analytical device. In particular, FIGS. 9A, 9B and 9C are the results of a paper-based PCR assay of Brucella genomic DNA on paper-based device with various layers (Number 1-16), and ddH2O as the negative control.

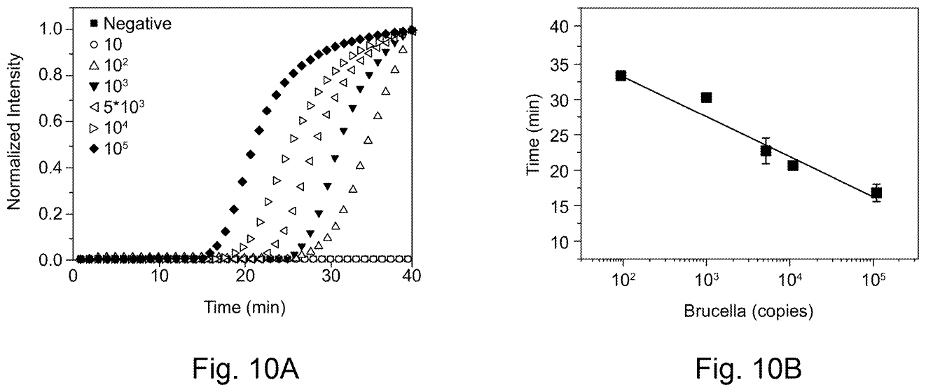

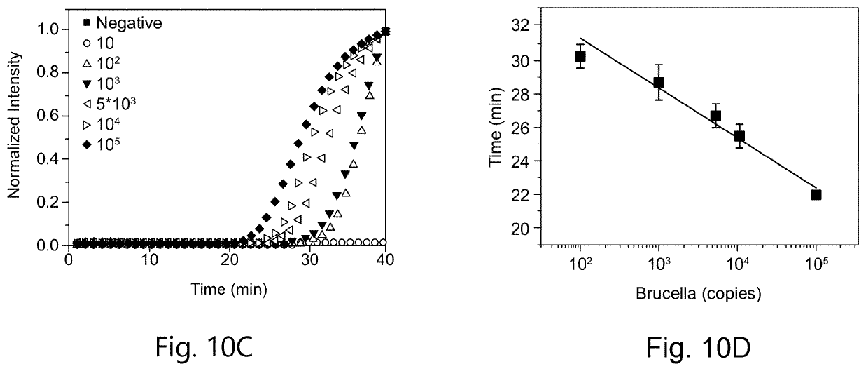

FIGS. 10A to 10D are graphs of a LAMP assay for the detection of model organism Brucella. In particular, FIGS. 10A and 10B are graphs of Real-time LAMP assay for Brucella detection. FIGS. 10C and 10D are graphs of paper-based LAMP assay for Brucella detection.

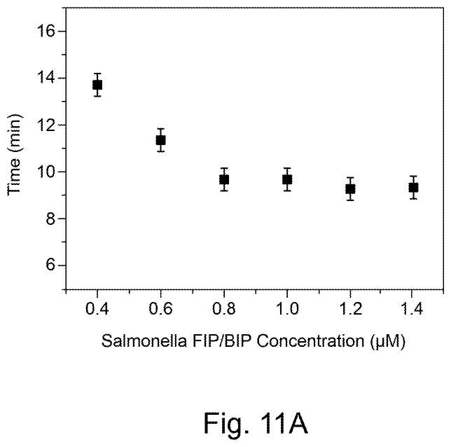

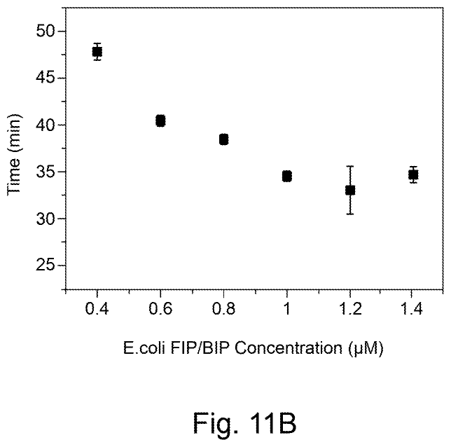

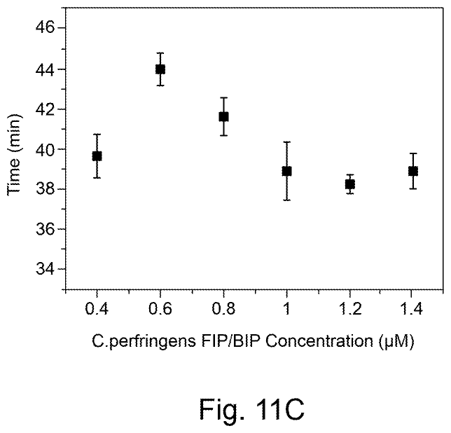

FIGS. 11A to 11C are graphs of the optimization of FIP/BIP concentration for LAMP assay on 3 genomic DNA targets. In particular, FIG. 11A is a graph of the results of a Real-time LAMP assay on Salmonella genomic DNA with various FIP/BIP concentration (0.4 μM; 0.6 μM; 0.8 μM; 1.0 μM; 1.2 μM; 1.4 μM), and ddH2O as the negative control. FIG. 11B is a graph of the results of a Real-time LAMP assay on E. coli genomic DNA with various FIP/BIP concentration (0.4 μM; 0.6 μM; 0.8 μM; 1.0 μM; 1.2 μM; 1.4 μM). FIG. 11C is a graph of the results of a Real-time LAMP assay on C. perfringens genomic DNA with various FIP/BIP concentration (0.4 μM; 0.6 μM; 0.8 μM; 1.0 μM; 1.2 μM; 1.4 μM).

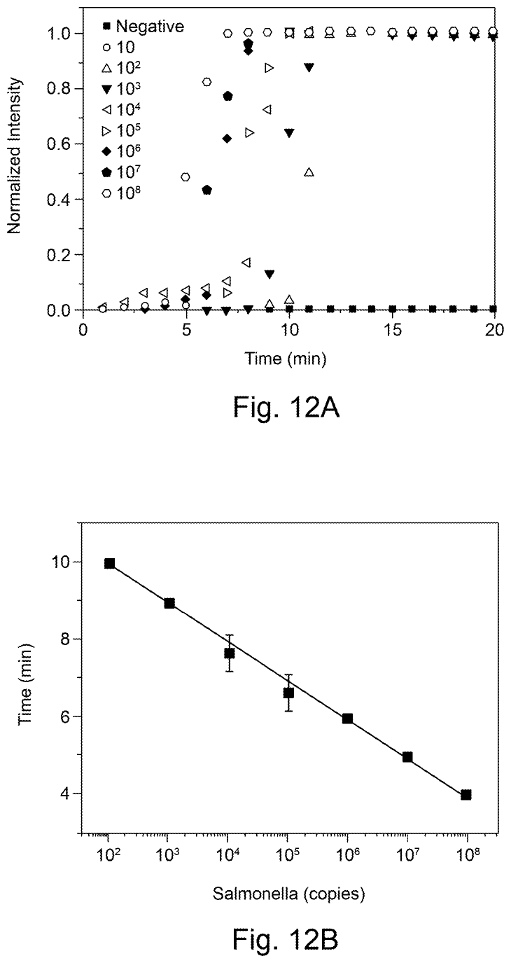

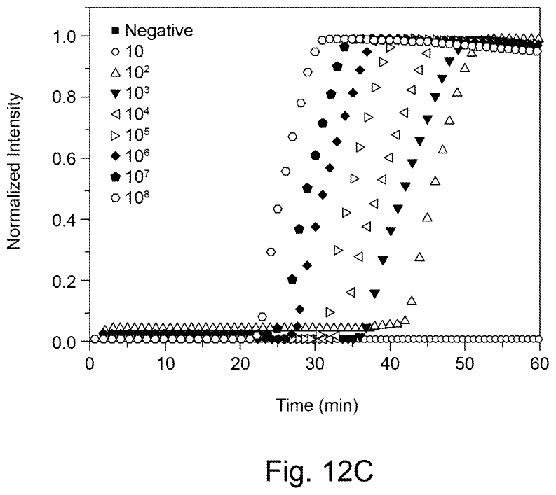

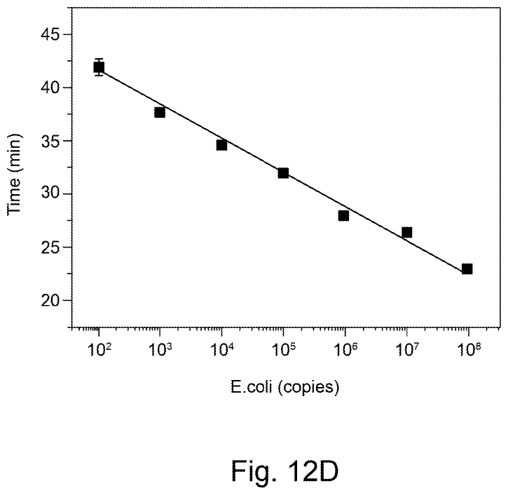

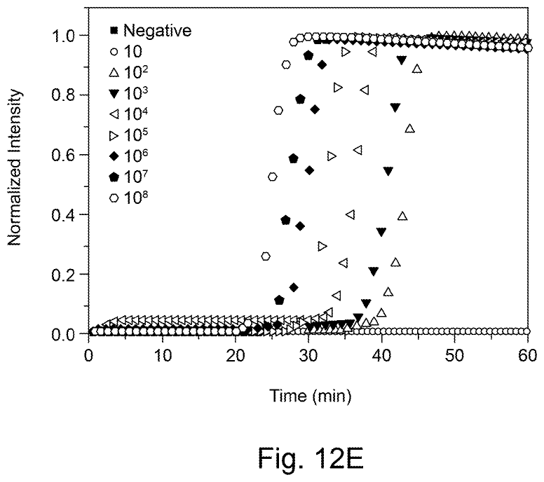

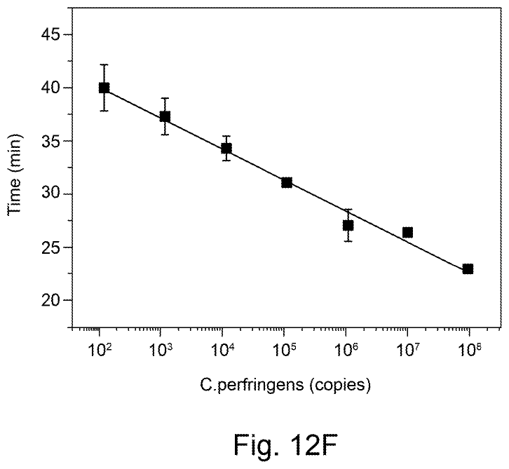

FIGS. 12A to 12F are graphs of the results of Real-time LAMP assay for the detection of 3 genomic DNA targets. In particular, FIGS. 12A and 12B are graphs of the results of a Real-time LAMP assay on Salmonella genomic DNA with various concentration (10 copies; 10² copies; 10³ copies; 10⁴ copies; 10⁵ copies; 10⁶ copies; 10⁷ copies; 10⁸ copies), and ddH2O as the negative control. FIGS. 12C and 12D are graphs of the results of a Real-time LAMP assay on E. coli genomic DNA with various concentration (10 copies; 10² copies; 10³ copies; 10⁴ copies; 10⁵ copies; 10⁶ copies; 10⁷ copies; 10⁸ copies). FIGS. 12E and 12F are graphs of the results of a Real-time LAMP assay on C. perfringens genomic DNA with various concentration (10 copies; 10² copies; 10³ copies; 10⁴ copies; 10⁵ copies; 10⁶ copies; 10⁷ copies; 10⁸ copies).

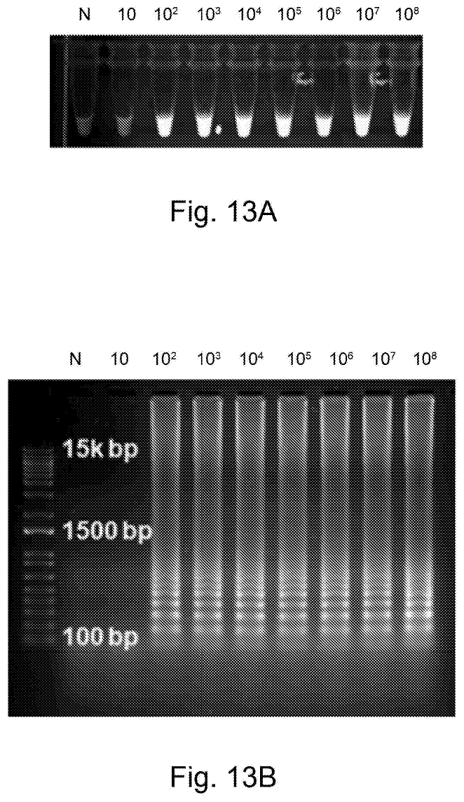

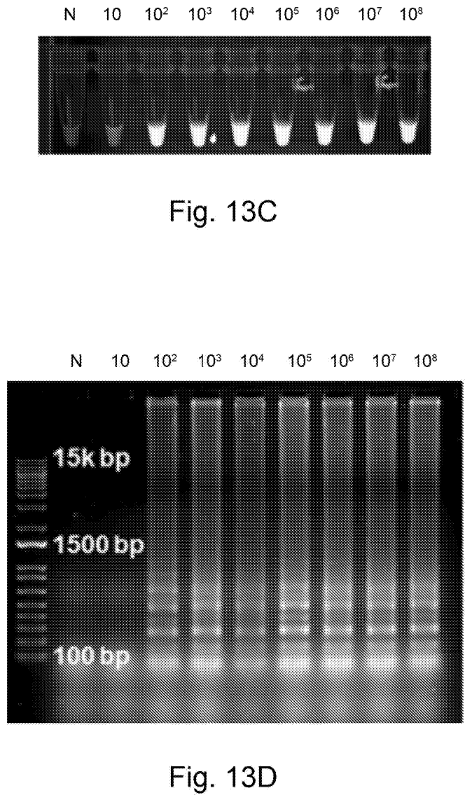

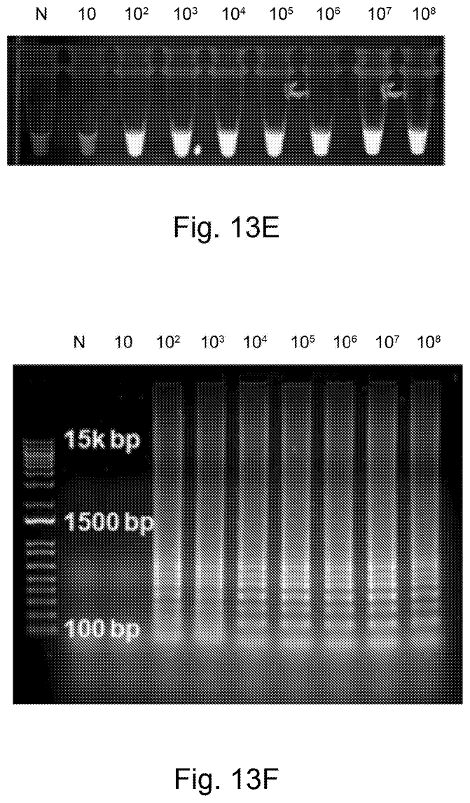

FIGS. 13A to 13F illustrate the detection of Real-time LAMP amplification products for the detection of 3 genomic DNA targets. In particular, FIG. 13A is a fluorescence image and FIG. 13B is an agarose gel image of Real-time LAMP assay on Salmonella genomic DNA with various concentration (10 copies; 10² copies; 10³ copies; 10⁴ copies; 10⁵ copies; 10⁶ copies; 10⁷ copies; 10⁸ copies), and ddH2O as the negative control. FIG. 13C is a fluorescence image and FIG. 13D is an agarose gel image of Real-time LAMP assay on E. coli genomic DNA with various concentration (10 copies; 10² copies; 10³ copies; 10⁴ copies; 10⁵ copies; 10⁶ copies; 10⁷ copies; 10⁸ copies). FIG. 13E is a fluorescence image and FIG. 13F is an agarose gel image of Real-time LAMP assay on C. perfringens genomic DNA with various concentration (10 copies; 10² copies; 10³ copies; 10⁴ copies; 10⁵ copies; 10⁶ copies; 10⁷ copies; 10⁸ copies).

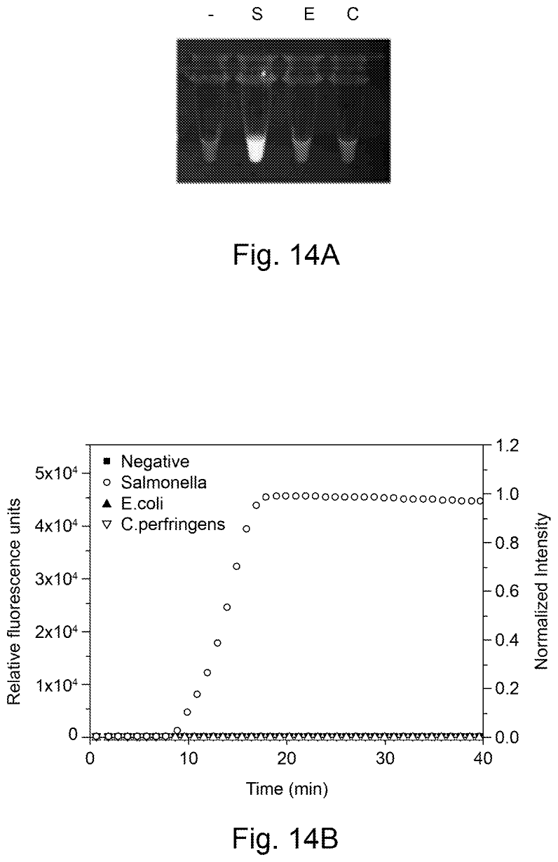

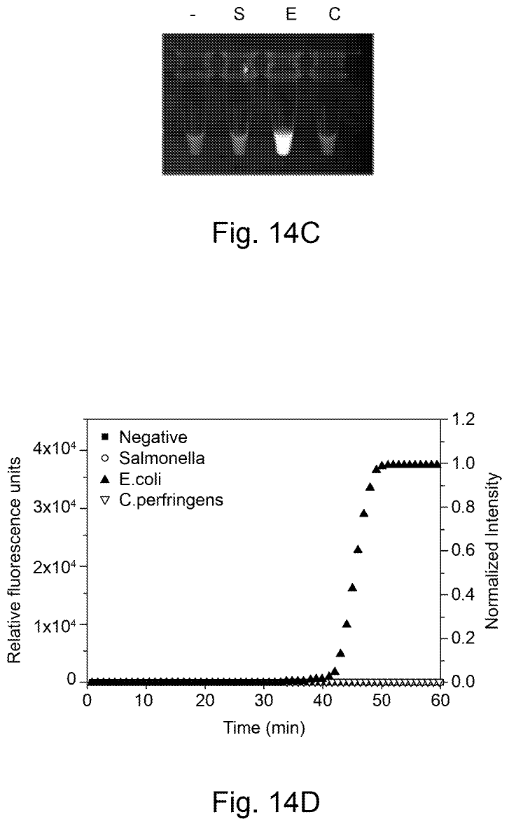

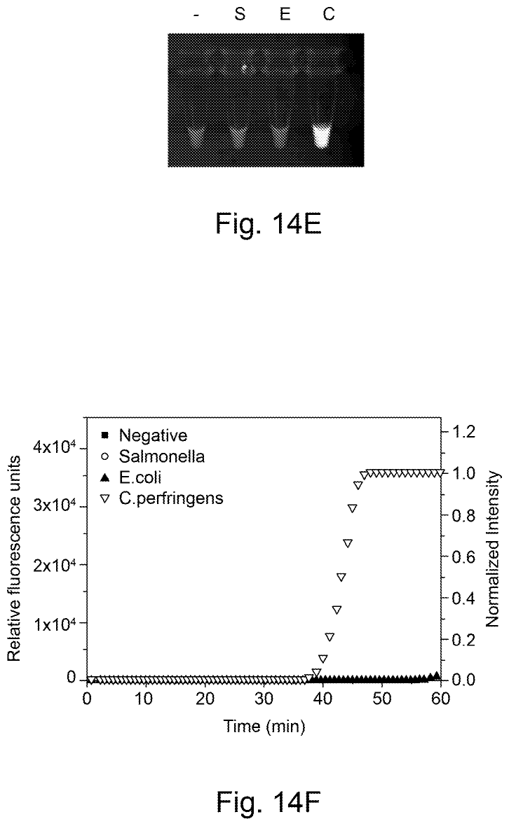

FIGS. 14A to 14F are graphs of the results of specificity of the primers sets for the detection of 3 genomic DNA targets. In particular, FIG. 14A is a fluorescence image and FIG. 14B is an amplification curve plot of Real-time LAMP assay with Salmonella primers set. FIG. 14C is a fluorescence image and FIG. 14D is an amplification curve plot of Real-time LAMP assay with E. coli primers set. FIG. 14E is a fluorescence image and FIG. 14F is an amplification curve plot of Real-time LAMP assay with C. perfringens primers set. refers to the negative control ddH2O; ‘S’ refers to Salmonella genomic DNA; ‘E’ refers to E. coli genomic DNA; ‘C’ refers to C. perfringens genomic DNA.

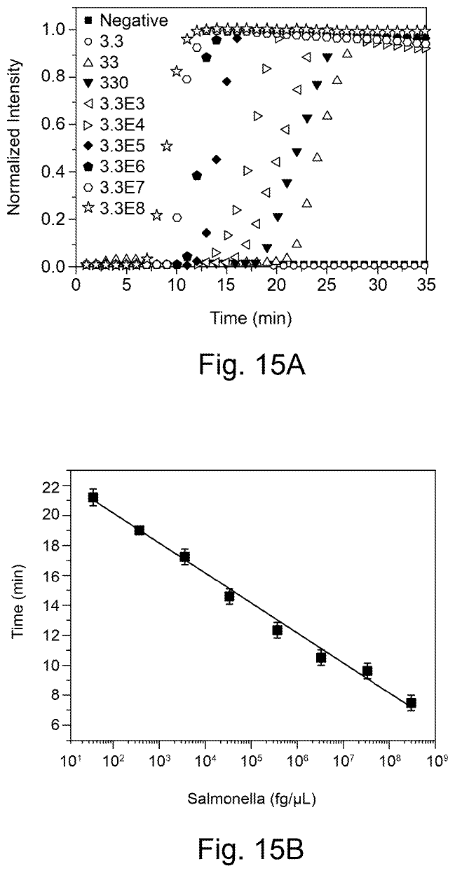

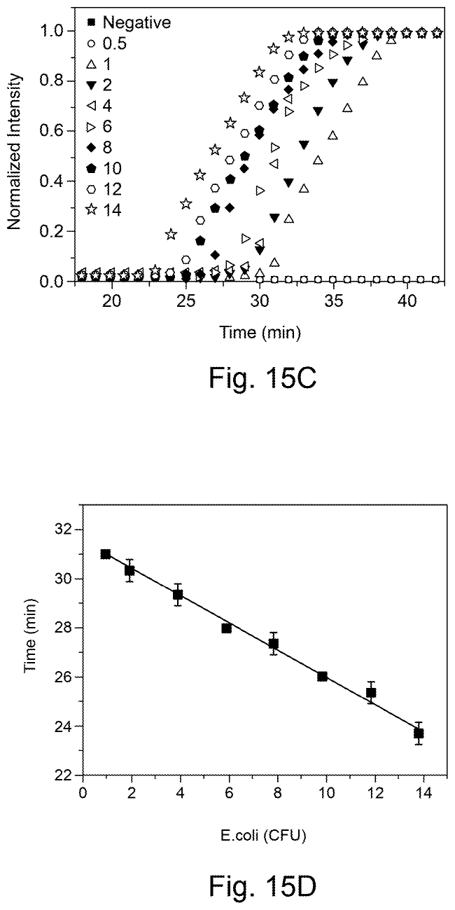

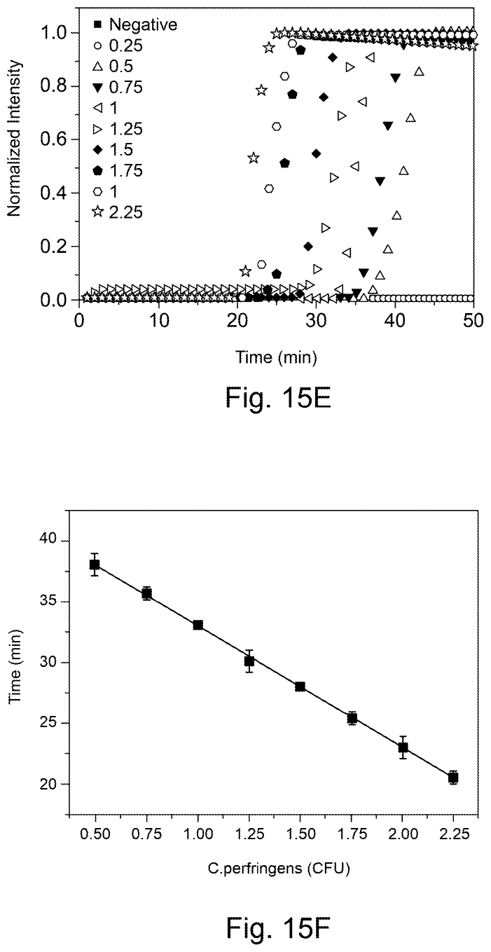

FIGS. 15A to 15F are graphs of the results of a paper-based LAMP assay for the detection of 3 organisms spiked into tap water. In particular, FIGS. 15A and 15B are graphs of the results of a paper-based LAMP assay on Salmonella genomic DNA with various concentration (3.3 fg μL⁻¹; 33 fg μL⁻¹; 330 fg μL⁻¹; 3.3 pg μL⁻¹; 33 pg μL⁻¹; 330 pg μL⁻¹; 3.3 ng μL⁻¹; 33 ng μL⁻¹; 330 ng μL⁻¹), and ddH2O as the negative control. FIGS. 15C and 15D are graphs of the results of paper-based LAMP assay on E. coli with various concentration (0.5 CFU, 1 CFU, 2 CFU, 4 CFU, 6 CFU, 8 CFU, 10 CFU, 12 CFU, 14 CFU). FIGS. 15E and 15F are graphs of the results of paper-based LAMP assay on C. perfringens with various concentration (0.25 CFU, 0.5 CFU, 0.75 CFU, 1 CFU, 1.25 CFU, 1.5 CFU, 1.75 CFU, 2 CFU, 2.25 CFU).

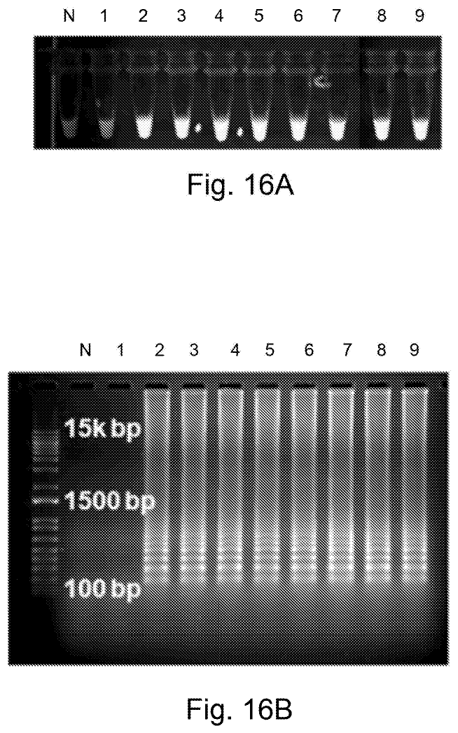

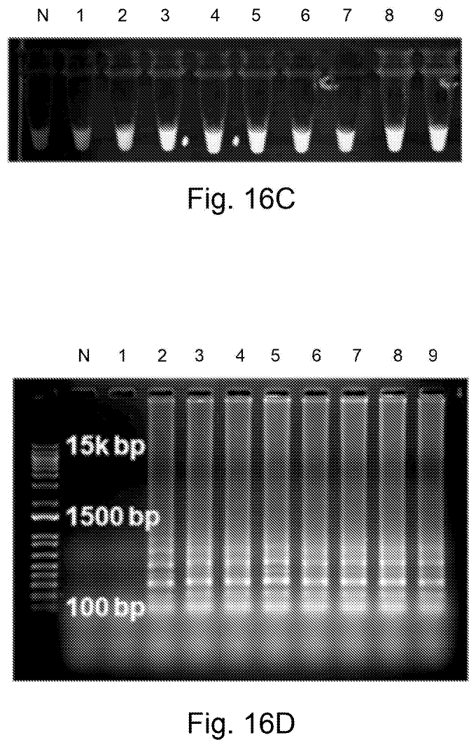

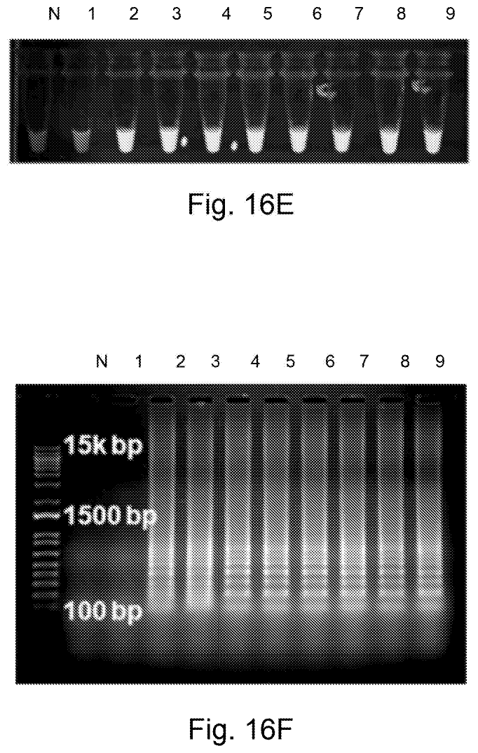

FIGS. 16A to 16F illustrate the detection of Paper-based LAMP amplification products for the detection of 3 organisms spiked into tap water. FIG. 16A is a fluorescence image and FIG. 16B is a agarose gel image of Paper-based LAMP assay on Salmonella genomic DNA with various concentration (1: 3.3 fg μL⁻¹; 2: 33 fg μL⁻¹; 3: 330 fg μL⁻¹; 4: 3.3 pg μL⁻¹; 5: 33 pg μL⁻¹; 6: 330 pg μL⁻¹; 7: 3.3 ng μL⁻¹; 8: 33 ng μL⁻¹; 9: 330 ng μL⁻¹), and ddH2O as the negative control. FIG. 16C is a fluorescence image and FIG. 16D is an agarose gel image of Paper-based LAMP assay on E. coli with various concentration (1: 0.5 CFU; 2: 1 CFU; 3: 2 CFU; 4: 4 CFU; 5: 6 CFU; 6: 8 CFU; 7: 10 CFU; 8: 12 CFU; 9: 14 CFU). FIG. 16E is a fluorescence image and FIG. 16F is an agarose gel image of Paper-based LAMP assay on C. perfringens with various concentration (1: 0.25 CFU; 2: 0.5 CFU; 3: 0.75 CFU; 4: 1 CFU: 5: 1.25 CFU; 6: 1.5 CFU; 7: 1.75 CFU; 8: 2 CFU; 9: 2.25 CFU).

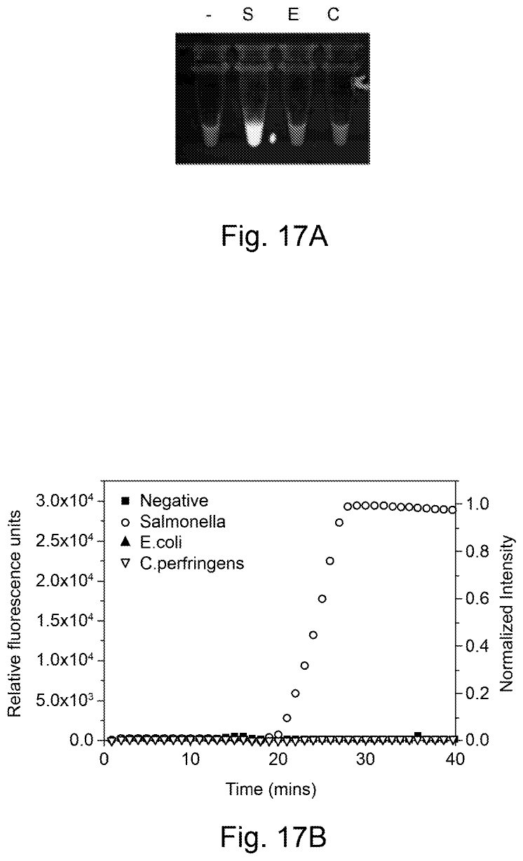

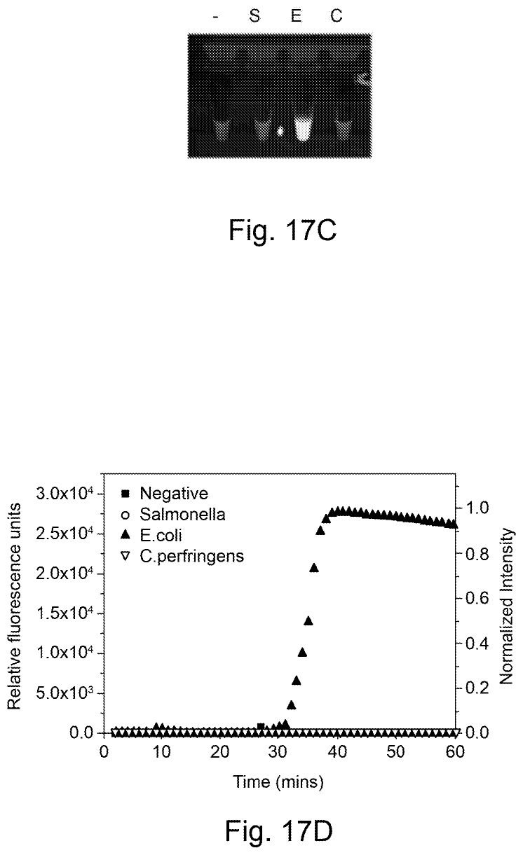

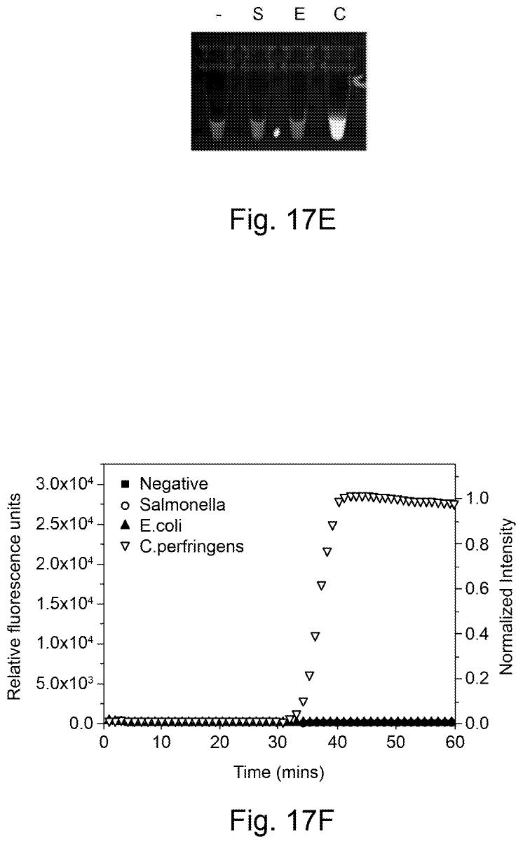

FIGS. 17A to 17F illustrate the specificity of the primers sets for the detection of 3 organisms spiked into tap water. FIG. 17A is a fluorescence image and FIG. 17B is an amplification curve plot of Paper-based LAMP assay with Salmonella primers sets. FIG. 17C is a fluorescence image and FIG. 17D is an amplification curve plot of Paper-based LAMP assay with E. coli primers sets. FIG. 17E is a fluorescence image and FIG. 17F is an amplification curve plot of Paper-based LAMP assay with C. perfringens primers sets. ‘-’ refers to the negative control ddH2O; S′ refers to Salmonella genomic DNA; ‘E’ refers to E. coli; C′ refers to C. perfringens.

The present μPAD enables a sample-to-answer assay within less than 1 hour. This was assessed using Salmonella, E. coli and C. perfringens, where different concentrations of organism samples were spiked into tap water (Salmonella at 3.3 fg μL⁻¹-330 ng E. coli at 0.5 CFU-14 CFU; C. perfringens at 0.25 CFU-2.25 CFU). The feasibility of Paper-based LAMP assay was assess as above and established the paper-based device could detect Salmonella genomic DNA, E. coli and C. perfringens as low as 33 fg 1 CFU and 0.5 CFU, respectively. The analytical specificity of the LAMP primers sets for detection of bacteria spiked into tap water was confirmed. LAMP products were detected for the associated targets, while no LAMP products were detected for the other targets.

The results indicate that the limits of detection of the present paper-based device are similar to those obtained in a real-time configuration on a Bio-Rad C1000 Thermal Cycler. LOD is defined as the target concentration that can be reliably detected as a positive signal by Paper-based LAMP assay. The device enabled an LOD of 33 fg μL⁻¹ for Salmonella determination, which is a 10-fold improvement in sensitivity compared with the LOD of 0.5 pg μL⁻¹ in an assay for Salmonella detection using a facile cascade signal-on colorimetric DNAzyme LAMP (dLAMP) sensor that integrates the LAMP technique and the inherent catalytic activity of the DNAzyme for simple target analysis. E. coli has been measured with the device at levels as low as 1 CFU, which is comparable to the LOD of 1 CFU based on a platform for E. coli detection by combining carbon nanotube (CNT) multilayer biosensors and the microfluidic chip-based LAMP technique. The limit of detection for C. perfringens was identified as 0.5 CFU. The present μPAD demonstrated a detection limit 20 times lower than that in a previous study for detection of C. perfringens in food with an LOD of 10 CFU mL⁻¹. Therefore, the present μPAD system and method has great potential for rapid detection of microbial/pathogen contamination in water or sewerage systems and networks especially in the resource-limited regions.

1. A method of detecting a pathogen present in a water sample comprising:
extracting nucleic acid of the pathogen from a nucleic acid solution derived from the water sample at a solid phase extraction structure mounted at a first layer of a multilayer device;
eluting nucleic acid from the solid phase extraction structure to at least a second layer of the multilayer device having paper-based fluid flow channels;
allowing the nucleic acid to flow thought the paper-based fluid flow channels to a further layer of the multilayer device having discrete reaction chambers, each of the chambers fed respectively by at least one of the fluid flow channels;
performing LAMP reactions within each reaction chamber to obtain LAMP products; and
detecting the LAMP products via an amplicon detection test.

2. The method as claimed in claim 1 further comprising filtering the water sample through a filter membrane and adding a lysis buffer to the filtered sample to form the DNA solution.

3. The method as claimed in claim 1 wherein prior to said step of eluting the nucleic acid, the method comprises washing the nucleic acid at the solid phase extraction structure with a washing buffer.

4. The method as claimed in claim 1 wherein the solid phase extraction structure comprises glass fibre.

5. The method as claimed in claim 1 wherein the step of allowing the nucleic acid to flow comprises allowing the nucleic acid to flow along the paper-based fluid flow channels of the second layer into paper-based fluid flow channels of a third layer positioned adjacent the second layer.

6. The method as claimed in claim 5 further comprising allowing the nucleic acid to flow from the paper-based fluid flow channels of the third layer into paper-based fluid flow channels of a fourth layer positioned adjacent the third layer.

7. The method as claimed in claim 1 wherein the flow of the nucleic acid in the paper-based fluid flow channels is divided as it transfers between the respective layers.

8. The method as claimed in claim 1 wherein the discrete reaction chambers at the further layer comprises paper inserts positioned within respective holes in the third layer, the further layer comprising a plastic material, optionally wherein prior to said step of performing the LAMP reactions, sealing the nucleic acid within the discrete reaction chambers by coating a film onto the further layer to cover the paper inserts within the holes.

9. (canceled)

10. The method as claimed in claim 1 wherein the step of performing the LAMP reactions comprises adding at least one set of LAMP primers to the discrete reaction chambers to create respective LAMP assays, optionally wherein the method further comprises adding a plurality of different sets of LAMP primers to the discrete reaction chambers, and optionally wherein the step of performing the LAMP reactions further comprises heating the further layer and the LAMP assays at a predetermined temperature and for a predetermined time.

11. (canceled)

12. (canceled)

13. The method as claimed in claim 10 wherein the step of performing the LAMP reactions further comprises heating the further layer and the LAMP assays at a predetermined temperature and for a predetermined time and wherein the predetermined temperature is in a range 40 to 80° C. and the predetermined time is in a range 10 to 90 minutes.

14. The method as claimed in claim 1 wherein the step of detecting the LAMP products comprises monitoring and capturing a signal from the LAMP products emitted from the reaction chambers, optionally wherein the step of capturing the signal comprises recording a fluorescent or UV signal as a photographic image, and optionally wherein the method further comprises analysing the at least one photographic image using software to obtain an average fluorescent or UV signal intensity of the LAMP products emitted from the respective reaction chambers.

15. (canceled)

16. (canceled)

17. The method as claimed in claim 1 wherein the step of detecting the LAMP products comprises using one of the discrete reaction chambers as an internal positive control containing a predetermined genomic nucleic acid as a template and using one of the discrete reaction chambers as an internal negative control containing a predetermined genomic nucleic acid as a template.

18. The method as claimed in claim 17, wherein the step of detecting the LAMP products comprises monitoring and capturing a signal from the LAMP products emitted from the reaction chambers, wherein the step of capturing the signal comprises recording a fluorescent or UV signal as a photographic image, wherein the method further comprises analysing the at least one photographic image using software to obtain an average fluorescent or UV signal intensity of the LAMP products emitted from the respective reaction chambers, and wherein the method further comprises normalising the average fluorescent or UV signal intensity of the LAMP products using an average fluorescent or UV signal intensity of the positive control and the negative control respectively.

19. A multilayer device for detecting a pathogen present in a water sample comprising:
a sample preparation part having at least one layer including a solid phase extraction structure mounted therein to receive a nucleic acid solution derived from the water sample;
a fluid flow part comprising a plurality of layers each having paper-based fluid flow channels therein to enable fluid capillary flow from the solid phase extraction structure through the plurality of layers;
a reaction layer comprising a plurality of discrete reaction chambers each provided in fluid communication with the fluid flow channels to receive by capillary flow a fluid from the fluid flow channels; and
optionally a filter membrane positioned in a fluid flow direction upstream of the solid phase extraction structure to enable a pre-filtering of the water sample and a nucleic acid lysising of the pathogen to form the nucleic acid solution.

20. (canceled)

21. The device as claimed in claim 19 wherein the plurality of layers of the fluid flow part comprises a plurality of primary layers each of the primary layers divided into a plurality of secondary layers, optionally wherein the primary layers and the secondary layers are integrally formed and coupled to one another by folded or hinge regions positioned at respective edges of the primary and secondary layers, and optionally wherein the fluid flow channels within each primary layer are divided respectively at the folded or hinge regions that divide respectively the primary layers into the secondary layers.

22. (canceled)

23. (canceled)

24. The device as claimed in claim 19 wherein the sample preparation part further comprises a layer having a sample introduction port and a layer having a waste collection component.

25. The device as claimed in claim 19 wherein the reaction layer comprises a plastic material having a plurality of holes and paper inserts positioned within the holes to define the discrete reaction chambers.

26. The device as claimed in claim 19 further comprising at least one set of LAMP primers for introduction to the discrete reaction chambers.

27. The device as claimed in claim 19 further comprising a lateral flow device having a plurality of lateral flow detection strips in fluid communication with the discrete reaction chambers respectively.

28. The device as claimed in any one of claim 19 further comprising a camera to capture an image of the discrete reaction chambers and software to analyse the image captured by the camera, optionally wherein the software is configured to analyse the images captured by the camera to determine an average fluorescent or UV signal intensity generated by the LAMP products derived from the LAMP primers.

29. (canceled)

30. (canceled)

31. (canceled)

32. (canceled)
